(19) United States
(12) Patent Application Publication
(10) Pub. No.: US 2024/0115591 A1
(43) Pub. Date: Apr. 11, 2024

(54) THERAPEUTIC METHODS AND COMPOSITIONS FOR TREATING BILIARY TRACT CANCER USING DEVIMISTAT

(71) Applicants: Cornerstone Pharmaceuticals, Inc., Cranbury, NJ (US); Regents of the University of Michigan, Ann Arbor, MI (US)

(72) Inventors: Sanjeev Luther, Cranbury, NJ (US); Timothy Pardee, Cranbury, NJ (US); Vaibhav Sahai, Ann Arbor, MI (US)

(21) Appl. No.: 18/251,521

(22) PCT Filed: Nov. 3, 2021
(86) PCT No.: PCT/US2021/057829

§ 371 Date: May 2, 2023

Related U.S. Application Data
(60) Provisional application No. 63/108,934, filed on Nov. 3, 2020.

(51) Int. Cl. A61K 31/7068; A61K 9/00; A61K 31/20; A61K 33/243; A61P 35/04

(52) U.S. Cl. CPC A61K 31/7068; A61K 9/0019; A61K 31/20; A61K 33/243; A61P 35/04

(57) ABSTRACT

The invention provides a method for treating biliary tract cancer in a patient in need thereof, comprising the step of intravenously administering to the patient a therapeutically effective amount of (i) devimistat, (ii) gemcitabine, and (iii) cisplatin, in order to treat the biliary tract cancer.

### CROSS-REFERENCE TO RELATED APPLICATION

This application claims the benefit of and priority to U.S. Provisional Patent Application Ser. No. 63/108,934, filed Nov. 3, 2020, the contents of which are hereby incorporated by reference in their entirety.

### FIELD OF THE INVENTION

The invention provides a method for treating biliary tract cancer in a patient in need thereof, comprising the step of intravenously administering to the patient a therapeutically effective amount of (i) devimistat, (ii) gemcitabine, and (iii) cisplatin, in order to treat the biliary tract cancer.

### BACKGROUND

Biliary tract cancer (BTC) develops as a result of malignant transformation of the biliary tract mucosa and is anatomically classified as intra-hepatic, extra-hepatic (hilar and distal) and gall bladder adenocarcinoma. BTC accounts for 10-15% of all primary liver cancer cases worldwide, and its incidence is rising (Shaib, Y. and H. B. El-Serag, “The epidemiology of cholangiocarcinoma,” Semin Liver Dis, 2004, 24(2), 115-125). Advanced BTCs are aggressive tumors with median survival time from diagnosis of less than 12 months (Valle, J., H. Wasan, D. H. Palmer, D. Cunningham, A. Anthoney, A. Maraveyas, S. Madhusudan, T. Iveson, S. Hughes, S. P. Pereira, M. Roughton, J. Bridgewater and A. B. C. T. Investigators, “Cisplatin plus gemcitabine versus gemcitabine for biliary tract cancer,” N Engl J Med, 2010, 362(14), 1273-1281), and five-year overall survival (OS) of ˜5% despite therapy (Nathan, H., T. M. Pawlik, C. L. Wolfgang, M. A. Choti, J. L. Cameron and R. D. Schulick, “Trends in survival after surgery for cholangiocarcinoma: A 30-year population-based SEER database analysis,” Journal of Gastrointestinal Surgery, 2007, 11, 1488-1497). The options for systemic chemotherapy for patients with advanced BTC remains limited with only a few meaningful improvements made over the past few decades. Valle et al randomly assigned 410 patients with locally advanced or metastatic BTC to receive gemcitabine with or without cisplatin in the phase III ABC-02 trial (Valle, Wasan et al. 2010). Patients on the gemcitabine cisplatin arm demonstrated an improvement in OS (11.7 versus 8.1 months; hazard ratio (HR), 0.64; 95% CI, 0.52 to 0.80; p<0.001) as compared to the gemcitabine alone arm. The objective response rate was 26.1% in the cisplatin and gemcitabine combination arm. The following clinically relevant grade 3 and 4 adverse events were noted in the gemcitabine-cisplatin arm: neutropenia (25.3%), anemia (7.6%), thrombocytopenia (15.7%), abnormal liver function (16.7%), fatigue (18.7%), nausea (4%), vomiting (5.1%), impaired renal function (1.5%), infection (18.2%), deep vein thrombosis (2%), and thromboembolic event (3.5%). This result established the gemcitabine 1000 mg/m² and cisplatin 25 mg/m² combination as a standard first line regimen for patients with advanced BTC.

Patients with advanced, unresectable or metastatic BTC have a poor prognosis despite systemic chemotherapy with gemcitabine and cisplatin. There is a great medical need for better therapies that not only have less toxicity but also the potential for greater efficacy. The present invention addresses this need and provides other related advantages.

### SUMMARY

The invention provides a method for treating biliary tract cancer in a patient in need thereof, comprising the step of intravenously administering to the patient a therapeutically effective amount of (i) devimistat, (ii) gemcitabine, and (iii) cisplatin, in order to treat the biliary tract cancer

The foregoing aspects of the invention are described in more detail, along with additional embodiments, in the detailed description below.

### BRIEF DESCRIPTION OF FIGURES

FIG. 1 depicts the effect of the devimistat dose at three simulated DLT probabilities on the predicted number of DLTs per dose per trial (upper left), number of treated patients per dose per trial (upper right), percent of trials with a given number of patients experiencing DLTs (lower left), and percent of trials that would select dose level −1, 1, 2, or 3 as the MTD or that would declare all levels too toxic and stop the trial.

### DETAILED DESCRIPTION

### I. Definitions

The term “devimistat” refers to 6,8-bis(benzylsulfanyl)octanoic acid (CPI-613), having the chemical structure

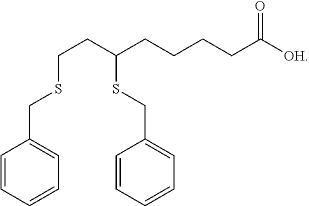 [SMILES: O=C(O)CCCCC(CCSCc1ccccc1)SCc1ccccc1]

As used herein, administering devimistat to a patient includes administering a pharmaceutically acceptable salt form of devimistat to the patient.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “overall response rate” or “ORR” refers to the sum of the complete response (CR) rate plus the partial response (PR) rate per RECIST version 1.1 criteria.

As used herein, the term “patient” refers to a human being in need of treatment for biliary tract cancer.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. A therapeutically effective amount can be determined experimentally in a laboratory or clinical setting, or may be the amount required by the guidelines of the United States Food and Drug Administration, or equivalent foreign agency, for the particular disease and patient being treated. It should be appreciated that determination of proper dosage forms, dosage amounts, and routes of administration is within the level of ordinary skill in the pharmaceutical and medical arts.

As used herein, the term “pharmaceutical composition” refers to the combination of an active agent (e.g., devimistat) with a pharmaceutically acceptable excipient, suitable for administration to a human being.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any pharmaceutical excipient suitable for use in humans. For examples of such excipients, see e.g., Martin, Remington's Pharmaceutical Sciences, 15th Ed., Mack Publ. Co., Easton, PA.

As used herein, the term “pharmaceutically acceptable salt” refers to any salt (e.g., acid or base) of a compound of the present invention which is suitable for administration to a human being. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NR 3, wherein R is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, IUPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified.

### II. Therapeutic Applications

The invention provides a method for treating biliary tract cancer in a patient in need thereof, comprising the step of intravenously administering to the patient a therapeutically effective amount of (i) devimistat, (ii) gemcitabine, and (iii) cisplatin, in order to treat the biliary tract cancer. The devimistat, gemcitabine, and cisplatin may be administered on the same or different days pursuant to any suitable cycle. Appropriate doses and cycles may be adapted from past experience with each agent in other cancer indications. For the convenience of the patient, it is preferred that the devimistat, gemcitabine, and cisplatin be administered on the fewest days of each cycle as possible, and for that reason it is preferred that the agents be administered on the same days of each cycle. In certain embodiments, the devimistat, gemcitabine, and cisplatin are all administered on the same days of each cycle. Any suitable cycle may be used. In certain embodiments, the cycle is 21 days. In certain embodiments, the devimistat, gemcitabine, and cisplatin are administered on days 1 and 8 of a 21 day cycle. In certain embodiments, the cycle is repeated at least once. In certain embodiments, the cycle is repeated at least five times. In certain embodiments, the cycle is repeated and treatment continues for at least one year. In certain embodiments, the cycle is repeated and treatment continues until the patient experiences disease progression, inter-current illness that prevents further treatment, or an unacceptable adverse event. In certain embodiments, the cycle is repeated and treatment continues until the patient experiences disease progression or an unacceptable adverse event.

### Type of Biliary Tract Cancer

The method may be further characterized according to the type of biliary tract cancer treated. In certain embodiments, the biliary tract cancer is metastatic biliary tract cancer. In certain embodiments, the biliary tract cancer is locally advanced. In certain embodiments, the biliary tract cancer is an intrahepatic bile duct cancer or an extrahepatic bile duct cancer. In certain embodiments, the biliary tract cancer is an intrahepatic bile duct cancer. In certain embodiments, the biliary tract cancer is an extrahepatic bile duct cancer. In certain embodiments, the biliary tract cancer is a perihilar (a.k.a. hilar) bile duct cancer. In certain embodiments, the biliary tract cancer is a distal bile duct cancer. In certain embodiments, the biliary tract cancer is a cancer of the gallbladder. In certain embodiments, the biliary tract cancer is an adenocarcinoma. In certain embodiments, the biliary tract cancer is advanced and unresectable. In certain embodiments, the biliary tract cancer is pathologically or cytologically confirmed carcinoma of the biliary tract that is not eligible for curative resection, transplantation, or ablative therapies. In certain embodiments, the biliary tract cancer is previously untreated with systemic treatments (chemotherapy or targeted therapy) for advanced biliary tract cancer.

### Devimistat

In the method of the present invention, the devimistat is intravenously administered to the patient as a pharmaceutical composition comprising devimistat and a pharmaceutically acceptable excipient. Any suitable pharmaceutical composition may be used. The devimistat may be formulated using the free acid or a salt thereof.

In certain embodiments, the pharmaceutical composition comprises a pharmaceutically acceptable salt of devimistat and a pharmaceutically acceptable excipient. In certain embodiments, the pharmaceutical composition comprises devimistat and a pharmaceutically acceptable excipient capable of forming an ion pair with devimistat. In certain embodiments, the pharmaceutical composition comprises devimistat and a pharmaceutically acceptable excipient capable of forming an ion pair with devimistat, wherein the pharmaceutically acceptable excipient comprises an aqueous solution of an ion pairing agent. Exemplary ion pairing agents include, for example, tertiary amines (such as triethanolamine), other amines such as diethanolamine, monoethanolamine, mefenamic acid and tromethamine, and combinations thereof. Other suitable ion pairing agents include organic Bronsted bases, alkali metal hydroxides, and alkaline earth metal hydroxides, such as, for example, cesium hydroxide. Additional exemplary ion pairing agents that may be used to prepare salts of devimistat include, for example, monoalkylamines, dialkylamines, trialkylamines, amino-substituted aliphatic alcohols, hydroxymonoalkylamines, hydroxydialkylamines, hydroxytrialkylamines, amino-substituted heteroaliphatic alcohols, alkyldiamines, substituted alkyldiamines, optionally substituted heteroaryl group containing at least one ring nitrogen atom, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. Additional ion pairing agents that may be used to prepare salts of devimistat include diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, and ammonium hydroxide.

In certain embodiments, the pharmaceutical composition comprises devimistat and triethanolamine. In certain embodiments, the pharmaceutical composition comprises devimistat and an aqueous solution of triethanolamine. In certain embodiments, the pharmaceutical composition of devimistat comprises a 50 mg/mL solution of devimistat in 1M aqueous triethanolamine.

In certain embodiments, the pharmaceutical composition is diluted with dextrose and water prior to intravenous administration. In certain embodiments, the pharmaceutical composition of devimistat comprises a 50 mg/mL solution of devimistat in 1M aqueous triethanolamine, which is diluted from 50 mg/mL to about 12.5 mg/mL with sterile aqueous 5% dextrose for injection (D5W) prior to administration to the patient. In certain embodiments, the pharmaceutical composition of devimistat comprises a 50 mg/mL solution of devimistat in 1M triethanolamine, and each 1 mL thereof is diluted with about 3 mL D5W prior to administration to a patient. In certain embodiments, the D5W-diluted solution of devimistat has a pH of about 8.4 to about 8.8. Preferably, the D5W-diluted solution of devimistat is stable for at least 24 hours. In certain embodiments, the D5W-diluted devimistat is administered by IV infusion at the rate of about 4 mL/min on day 1 and day 8 of each 21-day cycle. In certain embodiments, the D5W-diluted devimistat is administered by an IV infusion lasting about two hours on each of days 1 and 8 of a 21-day cycle. In certain embodiments, the devimistat infusion is administered concurrently with D5W. Preferably, the diluted devimistat pharmaceutical composition is administered concurrently with D5W, wherein the concurrent D5W is administered at the rate of about 125 mL/hour.

Preferably, the pharmaceutical composition of devimistat is administered via a central venous catheter. Preferably, the pharmaceutical composition of devimistat is administered via an infusion pump.

In certain embodiments, the devimistat used to prepare the pharmaceutical composition has a purity of at least about 90% (w/w). In certain embodiments, the devimistat used to prepare the pharmaceutical composition has a purity of at least about 95% (w/w). In certain embodiments, the devimistat used to prepare the pharmaceutical composition has a purity of at least about 98% (w/w). In certain embodiments, the devimistat used to prepare the pharmaceutical composition has a purity of at least about 99% (w/w). In certain embodiments, the devimistat used to prepare the pharmaceutical composition has a purity of at least about 99.3% (w/w). In certain embodiments, the devimistat used to prepare the pharmaceutical composition has a purity of at least about 99.5% (w/w).

The therapeutic method may be further characterized according to the dose of devimistat administered to the patient. The amount of devimistat to be administered is based on the body surface area (BSA) of the patient in square meters (m 2). In certain embodiments, the devimistat is administered at a dose that is at or near its maximum tolerated dose (MTD) when administered in combination with therapeutically effective amounts of gemcitabine and cisplatin. A suitable clinical trial for determining the MTD of devimistat is described in Example 1, below. In certain embodiments, the devimistat is intravenously administered to the patient at a dose of about 500 mg/m² to about 3000 mg/m² on each day the devimistat is administered to the patient (e.g., on each of days 1 and 8 of a 21 day cycle). In certain embodiments, the devimistat is intravenously administered to the patient at a dose of about 500 mg/m² to about 2000 mg/m² on each day the devimistat is administered to the patient (e.g., on each of days 1 and 8 of a 21 day cycle). In certain embodiments, the devimistat is intravenously administered to the patient at a dose of about 500 mg/m² on each day the devimistat is administered to the patient (e.g., on each of days 1 and 8 of a 21 day cycle). In certain embodiments, the devimistat is intravenously administered to the patient at a dose of about 1000 mg/m² on each day the devimistat is administered to the patient (e.g., on each of days 1 and 8 of a 21 day cycle). In certain embodiments, the devimistat is intravenously administered to the patient at a dose of about 1500 mg/m² on each day the devimistat is administered to the patient (e.g., on each of days 1 and 8 of a 21 day cycle). In certain embodiments, the devimistat is intravenously administered to the patient at a dose of about 2000 mg/m² on each day the devimistat is administered to the patient (e.g., on each of days 1 and 8 of a 21 day cycle). In certain embodiments, the devimistat is intravenously administered to the patient at a dose of about 2500 mg/m² on each day the devimistat is administered to the patient (e.g., on each of days 1 and 8 of a 21 day cycle). In certain embodiments, the devimistat is intravenously administered to the patient at a dose of about 3000 mg/m² on each day the devimistat is administered to the patient (e.g., on each of days 1 and 8 of a 21 day cycle). In the 21-day cycle, devimistat is preferably administered on days 1 and 8 prior to administration of gemcitabine and cisplatin. The administered dose of devimistat may be adjusted during treatment. For example, the administered dose of devimistat may be reduced if the patient experiences an adverse event. Dose adjustments suitable for use in the method of the present invention are described in Example 1, below.

In certain embodiments, the devimistat is administered intravenously to the patient in the form of a pharmaceutical composition comprising devimistat, water, and triethanolamine. In certain embodiments, the pharmaceutical composition comprises devimistat at a concentration of 50 mg/mL and triethanolamine at a concentration of 1M. In certain embodiments, the pharmaceutical composition further comprises dextrose.

### Gemcitabine

In the method of the present invention the gemcitabine (chemical name 2′-deoxy-2′,2′-difluorocytidine (β-isomer)) is intravenously administered to the patient as a pharmaceutical composition comprising gemcitabine and a pharmaceutically acceptable excipient. The gemcitabine may be formulated using the free base or a salt thereof. Preferably, the gemcitabine is intravenously administered as the HCl salt. Any suitable pharmaceutical composition may be used. Preferably, a commercially available pharmaceutical composition of gemcitabine is used. Gemcitabine is commercially available as a lyophilized powder in 200 mg and 1 g single-dose vials containing 200 mg gemcitabine (equivalent to 227.7 mg gemcitabine hydrochloride) and 1 g gemcitabine (equivalent to 1.139 g gemcitabine hydrochloride), respectively. The powder may be reconstituted with 5 mL (200 mg vial) or 25 mL (1 g vial) of 0.9% sodium chloride injection, USP to yield a solution. Prior to intravenous administration, the appropriate dose of gemcitabine is preferably withdrawn from the vial and then diluted with additional 0.9% sodium chloride injection, USP to a final concentration of at least 0.1 mg/mL.

The amount of gemcitabine to be administered is based on the body surface area (BSA) of the patient in square meters (m 2). A suitable gemcitabine dose may be determined with reference to the FDA-approved prescribing information for gemcitabine, and to prior experience with gemcitabine, including experience treating the same and similar indications. In certain embodiments, the gemcitabine is intravenously administered to the patient at a dose of about 500 mg/m² to about 1500 mg/m² on each day the gemcitabine is administered to the patient (e.g., on each of days 1 and 8 of a 21 day cycle). In certain embodiments, the gemcitabine is intravenously administered to the patient at a dose of about 500 mg/m² to about 1250 mg/m² on each day the gemcitabine is administered to the patient (e.g., on each of days 1 and 8 of a 21 day cycle). In certain embodiments, the gemcitabine is intravenously administered to the patient at a dose of about 640 mg/m² to about 1000 mg/m² on each day the gemcitabine is administered to the patient (e.g., on each of days 1 and 8 of a 21 day cycle). In certain embodiments, the gemcitabine is intravenously administered to the patient at a dose of about 800 mg/m² to about 1000 mg/m² on each day the gemcitabine is administered to the patient (e.g., on each of days 1 and 8 of a 21 day cycle). In certain embodiments, the gemcitabine is intravenously administered to the patient at a dose of about 500 mg/m² on each day the gemcitabine is administered to the patient (e.g., on each of days 1 and 8 of a 21 day cycle). In certain embodiments, the gemcitabine is intravenously administered to the patient at a dose of about 640 mg/m² on each day the gemcitabine is administered to the patient (e.g., on each of days 1 and 8 of a 21 day cycle). In certain embodiments, the gemcitabine is intravenously administered to the patient at a dose of about 800 mg/m² on each day the gemcitabine is administered to the patient (e.g., on each of days 1 and 8 of a 21 day cycle). In certain embodiments, the gemcitabine is intravenously administered to the patient at a dose of about 1000 mg/m² on each day the gemcitabine is administered to the patient (e.g., on each of days 1 and 8 of a 21 day cycle). In certain embodiments, the gemcitabine is intravenously administered to the patient at a dose of about 1250 mg/m² on each day the gemcitabine is administered to the patient (e.g., on each of days 1 and 8 of a 21 day cycle). Preferably, the gemcitabine is intravenously administered to the patient at a dose of about 1000 mg/m² on days 1 and 8 of a 21 day cycle as an infusion of about 25-35 minutes. Preferably, the gemcitabine is intravenously administered immediately after administration of the devimistat. The administered dose of gemcitabine may be adjusted during treatment. For example, the administered dose of gemcitabine may be reduced if the patient experiences an adverse event. Dose adjustments suitable for use in the method of the present invention are described in Example 1, below, and also in the FDA-approved prescribing information for gemcitabine.

In certain embodiments, the gemcitabine is administered intravenously to the patient in the form of a pharmaceutical composition comprising gemcitabine hydrochloride and water.

In certain embodiments, the gemcitabine is administered intravenously to the patient after devimistat has been administered intravenously to the patient.

### Cisplatin

In the method of the present invention the cisplatin (chemical name cis-diaminedichloroplatinum) is intravenously administered to the patient as a pharmaceutical composition comprising cisplatin and a pharmaceutically acceptable excipient. Any suitable pharmaceutical composition may be used. Preferably, a commercially available pharmaceutical composition of cisplatin is used. Cisplatin is commercially available as a lyophilized powder in 50 mg single-dose vials. The lyophilized powder may be reconstituted with 50 mL of sterile water for injection to yield a solution containing 1 mg/mL cisplatin. Prior to intravenous administration, the appropriate dose of cisplatin is preferably withdrawn from the vial and then diluted with about 1-2 L of a compatible infusion solution.

The amount of cisplatin to be administered is based on the body surface area (BSA) of the patient in square meters (m 2). A suitable cisplatin dose may be determined with reference to the FDA-approved prescribing information for cisplatin, and to prior experience with cisplatin in the same and similar indications. In certain embodiments, the cisplatin is intravenously administered to the patient at a dose of about 25 mg/m² on each day the cisplatin is administered to the patient (e.g., on each of days 1 and 8 of a 21 day cycle). In certain embodiments, the cisplatin is intravenously administered to the patient at a dose of about 20 mg/m² on each day the cisplatin is administered to the patient (e.g., on each of days 1 and 8 of a 21 day cycle). In certain embodiments, the cisplatin is intravenously administered to the patient at a dose of about 16 mg/m² on each day the cisplatin is administered to the patient (e.g., on each of days 1 and 8 of a 21 day cycle). Preferably, the cisplatin is intravenously administered to the patient at a dose of about 25 mg/m² on days 1 and 8 of a 21 day cycle as an infusion of about 25-65 minutes. Preferably, the cisplatin is intravenously administered immediately after administration of the gemcitabine. The administered dose of cisplatin may be adjusted during treatment. For example, the administered dose of cisplatin may be reduced if the patient experiences an adverse event. Dose adjustments suitable for use in the method of the present invention are described in Example 1, below, and also in the FDA-approved prescribing information for cisplatin.

In certain embodiments, the cisplatin is administered intravenously to the patient after gemcitabine has been administered intravenously to the patient.

### Patients for Treatment

Patients treated pursuant to the method of the present invention preferably meet at least one of the following eligibility criteria: a) pathologically or cytologically confirmed carcinoma of the biliary tract that is not eligible for curative resection, transplantation, or ablative therapies; b) ≥18 years of age; c) Eastern Cooperative Oncology Group (ECOG) Performance status of 0-1; d) adequate organ function within 2 weeks prior to treatment. In certain embodiments, patients treated pursuant to the method of the present invention meet at least a) and b) of the above eligibility criteria. In certain embodiments, patients treated pursuant to the method of the present invention meet all of the above eligibility criteria. With respect to eligibility criterion d), adequate organ function may be evidenced by: i) absolute neutrophil count [ANC]≥1500/mm³; ii) hemoglobin ≥9 g/dL; iii) platelets ≥100,000/mm³; iv) serum creatinine ≤1.5×ULN; v) creatinine clearance ≥50 mL/min; vi) albumin ≥3.0 g/dL; vii) AST/ALT≤3.0×UNL (≤5×UNL if liver metastasis); viii) total bilirubin ≤1.5×UNL; and ix) INR≤1.5×UNL.

### IV. Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial in which the method of the present invention is tested in one arm and standard therapy with gemcitabine and cisplatin is tested in a second arm, the method of the present invention preferably provides a median overall survival (OS) of at least three months. In certain embodiments, the phase II or phase III trial is conducted as described in Example 1. Preferably, the phase II or phase III clinical trial comprises at least 30 patients. More preferably, the phase II or phase III clinical trial comprises at least 40 patients. More preferably, the phase II or phase III clinical trial comprises at least 50 patients. More preferably, the phase II or phase III clinical trial comprises at least 75 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, when tested in such a phase II or phase III clinical trial the method of the present invention provides an OS of at least 4 months. More preferably, the OS is at least 5 months. More preferably, the OS is at least 6 months. More preferably, the OS is at least 7 months. More preferably, the OS is at least 8 months. More preferably, the OS is at least 9 months. More preferably, the OS is at least 10 months. More preferably, the OS is at least 11 months. More preferably, the OS is at least 12 months. More preferably, the OS is at least 13 months. More preferably, the OS is at least 14 months. More preferably, the OS is at least 15 months. More preferably, the OS is at least 16 months. More preferably, the OS is at least 17 months. More preferably, the OS is at least 18 months. More preferably, the OS is at least 19 months. More preferably, the OS is at least 20 months. More preferably, the OS is at least 21 months. More preferably, the OS is at least 22 months. More preferably, the OS is at least 23 months. More preferably, the OS is at least 24 months. Preferably, when tested in such a phase II or phase III clinical trial the method of the present invention provides an OS higher than the control (standard gemcitabine-cisplatin) arm. Preferably, the OS is at least 1 month longer than the control arm (e.g., if the OS of the patients in the control arm of the clinical trial is 4 months, 7 months, or 11 months, then the OS of the patients in the clinical trial treated according to the method of the present invention is ≥5 months, ≥8 months, or ≥12 months, respectively). More preferably, the OS is at least 2 months longer than the control arm. More preferably, the OS is at least 3 months longer than the control arm. More preferably, the OS is at least 4 months longer than the control arm. More preferably, the OS is at least 5 months longer than the control arm. More preferably, the OS is at least 6 months longer than the control arm. More preferably, the OS is at least 7 months longer than the control arm. More preferably, the OS is at least 8 months longer than the control arm. More preferably, the OS is at least 9 months longer than the control arm. More preferably, the OS is at least 10 months longer than the control arm. More preferably, the OS is at least 11 months longer than the control arm. More preferably, the OS is at least 12 months longer than the control arm. More preferably, the OS is at least 13 months longer than the control arm. More preferably, the OS is at least 14 months longer than the control arm. More preferably, the OS is at least 15 months longer than the control arm. More preferably, the OS is at least 16 months longer than the control arm. More preferably, the OS is at least 17 months longer than the control arm. More preferably, the OS is at least 18 months longer than the control arm. Preferably, when tested in such a phase II or phase III clinical trial, the method of the present invention provides an OS longer than the historical OS level, i.e., the OS observed in previous clinical trials testing treatment of similar patients with a similar regimen of gemcitabine-cisplatin alone. Preferably, the OS is at least 2 months longer than the historical OS level (e.g., if the historical OS level is 5 months, then the OS of the patients in the clinical trial treated according to the method of the present invention is at least 7 months). More preferably, the OS is at least 3 months longer than the historical OS level. More preferably, the OS is at least 4 months longer than the historical OS level. More preferably, the OS is at least 5 months longer than the historical OS level. More preferably, the OS is at least 6 months longer than the historical OS level. More preferably, the OS is at least 7 months longer than the historical OS level. More preferably, the OS is at least 8 months longer than the historical OS level. More preferably, the OS is at least 9 months longer than the historical OS level. More preferably, the OS is at least 10 months longer than the historical OS level. More preferably, the OS is at least 11 months longer than the historical OS level. More preferably, the OS is at least 12 months longer than the historical OS level. More preferably, the OS is at least 13 months longer than the historical OS level. More preferably, the OS is at least 14 months longer than the historical OS level. More preferably, the OS is at least 15 months longer than the historical OS level. More preferably, the OS is at least 16 months longer than the historical OS level. More preferably, the OS is at least 17 months longer than the historical OS level. More preferably, the OS is at least 18 months longer than the historical OS level.

Preferably, when tested in a phase II or phase III clinical trial as described herein, the method of the present invention provides a median progression free survival (PFS) of at least three months. More preferably, the method of the present invention provides PFS of at least 4 months. More preferably, the PFS is at least 5 months. More preferably, the PFS is at least 6 months. More preferably, the PFS is at least 7 months. More preferably, the PFS is at least 8 months. More preferably, the PFS is at least 9 months. More preferably, the PFS is at least 10 months. More preferably, the PFS is at least 11 months. More preferably, the PFS is at least 12 months. More preferably, the PFS is at least 13 months. More preferably, the PFS is at least 14 months. More preferably, the PFS is at least 15 months. More preferably, the PFS is at least 16 months. More preferably, the PFS is at least 17 months. More preferably, the PFS is at least 18 months. More preferably, the PFS is at least 19 months. More preferably, the PFS is at least 20 months. More preferably, the PFS is at least 21 months. More preferably, the PFS is at least 22 months. More preferably, the PFS is at least 23 months. More preferably, the PFS is at least 24 months. Preferably, the PFS is longer than the control (standard gemcitabine-cisplatin) arm, i.e., the PFS of the patients in the clinical trial treated according to the method of the present invention is longer than the PFS of the patients in the control (standard gemcitabine-cisplatin) arm of the clinical trial. Preferably, the PFS is at least 1 month longer than the control arm (e.g., if the PFS of the patients in the control arm of the clinical trial is 4 months, 7 months, or 11 months, then the PFS of the patients in the clinical trial treated according to the method of the present invention is ≥5 months, ≥8 months, or ≥12 months, respectively). More preferably, the PFS is at least 2 months longer than the control arm. More preferably, the PFS is at least 3 months longer than the control arm. More preferably, the PFS is at least 4 months longer than the control arm. More preferably, the PFS is at least 5 months longer than the control arm. More preferably, the PFS is at least 6 months longer than the control arm. More preferably, the PFS is at least 7 months longer than the control arm. More preferably, the PFS is at least 8 months longer than the control arm. More preferably, the PFS is at least 9 months longer than the control arm. More preferably, the PFS is at least 10 months longer than the control arm. More preferably, the PFS is at least 11 months longer than the control arm. More preferably, the PFS is at least 12 months longer than the control arm. More preferably, the PFS is at least 13 months longer than the control arm. More preferably, the PFS is at least 14 months longer than the control arm. More preferably, the PFS is at least 15 months longer than the control arm. More preferably, the PFS is at least 16 months longer than the control arm. More preferably, the PFS is at least 17 months longer than the control arm. More preferably, the PFS is at least 18 months longer than the control arm. Preferably, when tested in a phase II or phase III clinical trial as described herein, the method of the present invention provides a PFS longer than the historical PFS level, i.e., the PFS observed in previous clinical trials testing treatment of similar patients with a similar regimen of gemcitabine-cisplatin alone. Preferably, the PFS is at least 1 month longer than the historical PFS level (e.g., if the historical PFS level is 5 months, then the PFS of the patients in the clinical trial treated according to the method of the present invention is at least 6 months). More preferably, the PFS is at least 2 months longer than the historical PFS level. More preferably, the PFS is at least 3 months longer than the historical PFS level. More preferably, the PFS is at least 4 months longer than the historical PFS level. More preferably, the PFS is at least 5 months longer than the historical PFS level. More preferably, the PFS is at least 6 months longer than the historical PFS level. More preferably, the PFS is at least 7 months longer than the historical PFS level. More preferably, the PFS is at least 8 months longer than the historical PFS level. More preferably, the PFS is at least 9 months longer than the historical PFS level. More preferably, the PFS is at least 10 months longer than the historical PFS level. More preferably, the PFS is at least 11 months longer than the historical PFS level. More preferably, the PFS is at least 12 months longer than the historical PFS level. More preferably, the PFS is at least 13 months longer than the historical PFS level. More preferably, the PFS is at least 14 months longer than the historical PFS level. More preferably, the PFS is at least 15 months longer than the historical PFS level. More preferably, the PFS is at least 16 months longer than the historical PFS level. More preferably, the PFS is at least 17 months longer than the historical PFS level. More preferably, the PFS is at least 18 months longer than the historical PFS level.

Preferably, when tested in a phase II or phase III clinical trial as described herein, the method of the present invention provides an overall response rate (ORR) of at least 10%. More preferably, the ORR is at least 20%. More preferably, the ORR is at least 25%. More preferably, the ORR is at least 30%. More preferably, the ORR is at least 35%. More preferably, the ORR is at least 40%. More preferably, the ORR is at least 45%. More preferably, the ORR is at least 50%. More preferably, the ORR is at least 55%. More preferably, the ORR is at least 60%. More preferably, the ORR is at least 65%. More preferably, the ORR is at least 70%. More preferably, the ORR is at least 75%. More preferably, the ORR is at least 80%. More preferably, the ORR is at least 85%. More preferably, the ORR is at least 90%. More preferably, the ORR is at least 95%. Preferably, when tested in a phase II or phase III clinical trial as described herein, the method of the present invention provides an ORR higher than the historical ORR level, i.e., the ORR observed in previous clinical trials testing treatment of similar patients with a similar regimen of gemcitabine-cisplatin alone. Preferably, the ORR is at least 5% higher than the historical ORR level (e.g., if the historical ORR level is 25%, then the ORR of the patients in the clinical trial treated according to the method of the present invention is at least 30%). More preferably, the ORR is at least 10% higher than the historical ORR level. More preferably, the ORR is at least 15% higher than the historical ORR level. More preferably, the ORR is at least 20% higher than the historical ORR level. More preferably, the ORR is at least 25% higher than the historical ORR level. More preferably, the ORR is at least 30% higher than the historical ORR level. More preferably, the ORR is at least 35% higher than the historical ORR level. More preferably, the ORR is at least 40% higher than the historical ORR level. More preferably, the ORR is at least 45% higher than the historical ORR level. More preferably, the ORR is at least 50% higher than the historical ORR level. More preferably, the ORR is at least 55% higher than the historical ORR level. More preferably, the ORR is at least 60% higher than the historical ORR level. More preferably, the ORR is at least 65% higher than the historical ORR level. More preferably, the ORR is at least 70% higher than the historical ORR level. More preferably, the ORR is at least 75% higher than the historical ORR level. More preferably, the ORR is at least 80% higher than the historical ORR level. Preferably, the ORR is higher than the control (standard gemcitabine-cisplatin) arm, i.e., the ORR of the patients in the clinical trial treated according to the method of the present invention is higher than the ORR of the patients in the control (standard gemcitabine-cisplatin) arm of the clinical trial. Preferably, the ORR is at least 5% higher than the control arm (e.g., if the ORR of the control arm patients is 45%, 55%, or 70%, then the ORR of the patients in the clinical trial treated according to the method of the present invention is ≥50%, ≥60%, or ≥75%, respectively). More preferably, the ORR is at least 10% higher than the control arm. More preferably, the ORR is at least 15% higher than the control arm. More preferably, the ORR is at least 20% higher than the control arm. More preferably, the ORR is at least 25% higher than the control arm. More preferably, the ORR is at least 30% higher than the control arm. More preferably, the ORR is at least 35% higher than the control arm. More preferably, the ORR is at least 40% higher than the control arm. More preferably, the ORR is at least 45% higher than the control arm. More preferably, the ORR is at least 50% higher than the control arm. More preferably, the ORR is at least 55% higher than the control arm. More preferably, the ORR is at least 60% higher than the control arm. More preferably, the ORR is at least 65% higher than the control arm. More preferably, the ORR is at least 70% higher than the control arm. More preferably, the ORR is at least 75% higher than the control arm. More preferably, the ORR is at least 80% higher than the control arm.

### V. Medical Kits

Another aspect of the invention provides a medical kit containing a therapeutic agent and/or pharmaceutical composition described herein, along with instructions for using the kit to treat biliary tract cancer according the methods described herein. In certain embodiments, the medical kit comprises (i) devimistat, and (ii) instructions for treating biliary tract cancer in a patient with devimistat, gemcitabine, and cisplatin as described herein.

### VI. EXAMPLES

The invention now being generally described, will be more readily understood by reference to the following examples, which are included merely for purposes of illustration of certain aspects and embodiments of the present invention, and are not intended to limit the invention.

### Example 1—Treatment of Advanced Unresectable Biliary Tract Cancer (BTC) in Adult Human Patients Using a Combination of Devimistat, Gemcitabine, and Cisplatin

### Study Design

This is a multi-center randomized phase IB/II study of gemcitabine and cisplatin with or without devimistat as first line therapy for patient with advanced unresectable biliary tract cancer.

### Objectives

Primary Objectives:
Phase IB—Determine the maximum tolerated dose/recommended phase 2 dose for gemcitabine and cisplatin with devimistat
Phase II—Determine the overall response rate in patients with advanced BTC treated with gemcitabine and cisplatin with or without devimistat.

Secondary Objectives:
Evaluate the median PFS and OS of patients with advanced BTC.
Evaluate the safety of devimistat in combination with gemcitabine and cisplatin in this patient population.

Exploratory Objectives:
To explore predictors of biomarker response and mechanisms of resistance based on the exploratory analysis of tissue obtained through serial biopsies and blood.
   Immunohistochemical staining for PDK, PDH, KGDH, SOD2 and CD79a
   Whole exome genomic and transcriptomic (RNAseq) analysis for tumor biology at baseline and progression.
   Blood collection, including serum, plasma and serum for future biomarker analysis, including ctDNA.

Endpoints Assessment:
Primary Endpoint Assessment: Overall response rate (ORR) will be determined as per the RECISTv1.1 criteria.
Secondary Endpoint Assessment: The progression-free survival (PFS) will be defined as time from date of treatment to date of radiological or clinical progression (leading to withdrawal from the study), or death from any cause, whichever comes first. Follow-up time will be censored at the date of last disease evaluation. Overall survival (OS) will be defined from the date of treatment to either date of death or censoring. Adverse events and reportable serious events are defined by the study protocol (NCI Common Toxicity Criteria for Adverse Events (CTCAE) v5.0).

### Number of Subjects

68-78 subjects.

### Eligibility Criteria

1. Patients must have a pathologically or cytologically confirmed carcinoma (except neuroendocrine) of the biliary tract (intra-hepatic, extra-hepatic (hilar, distal) or gallbladder) that is not eligible for curative resection, transplantation, or ablative therapies. Tumors of mixed histology are excluded.
2. Patients must not have received prior systemic treatment (chemotherapy or targeted therapy) for advanced BTC. Prior adjuvant chemotherapy is permitted provided it was completed >6 months from enrollment.
3. Prior radiation, chemoembolization, radioembolization or other local ablative therapies or hepatic resection is permitted if completed ≥4 weeks prior to enrollment AND if patient has recovered to ≤grade 1 toxicity. Extrahepatic palliative radiation is permitted if completed ≥2 weeks prior to enrollment AND if patient has recovered to ≤grade 1 toxicity.
4. Patients must have radiographically measurable disease (as per RECISTv1.1) in at least one site not previously treated with radiation or liver directed therapy (including bland, chemo- or radio-embolization, or ablation) either within the liver or in a metastatic site.
5. Patients must be ≥18 years of age.
6. Patients must have an ECOG performance status of 0-1
7. Patients must have the ability to understand and willingness to sign IRB-approved informed consent.
8. Patients must be willing to provide archived tissue, if available, from a previous diagnostic biopsy or surgery.
9. Patients must be able to tolerate CT and/or MRI with contrast.
10. Patients must have adequate organ function obtained ≤2 weeks prior to enrollment (absolute neutrophil count >1500/mm³, hemoglobin >9 g/dL, platelets >100,000/mm³, serum creatinine <1.5× upper limit normal (ULN), creatinine clearance ≥50 mL/min, albumin >3.0 g/dL, AST/ALT<3.0×ULN (<5×ULN if liver metastasis), total bilirubin <1.5× upper limit normal, INR<1.5 upper limit normal).
11. Patients must not have prior history of brain metastasis (unless previously treated, asymptomatic and stable for at least 3 months), or organ transplantation.
12. Patients must not have undergone a major surgical procedure <4 weeks prior to enrollment.
13. Patients must not have an active second malignancy other than non-melanoma skin cancer or cervical carcinoma in situ. Patients with history of malignancy are eligible provided primary treatment of that cancer was completed >1 year prior to enrollment and the patient is free of clinical or radiologic evidence of recurrent or progressive malignancy.
14. Patients must have no ongoing active, uncontrolled infections (afebrile for >48 hours off antibiotics).
15. Patients must not have a psychiatric illness, other significant medical illness, or social situation which, in the investigator's opinion, would limit compliance or ability to comply with study requirements.
16. Women must not be pregnant or breastfeeding since study drugs may harm the fetus or child. All females of childbearing potential (not surgically sterilized and between menarche and 1-year post menopause) must have a negative screening pregnancy test.
17. Women of child-bearing potential and men must agree to use 2 methods of adequate contraception (hormonal plus barrier or 2 barrier forms) OR abstinence prior to study entry, for the duration of study participation, and for 6 months (for men and women) following completion of study therapy.
18. Patients must not have active heart disease including symptomatic heart failure (NYHA class 3 or 4), unstable angina pectoris, uncontrolled cardiac arrhythmia or interstitial lung disease.
19. Prisoners or subjects who are involuntarily incarcerated, or compulsorily detained for treatment of either a psychiatric or physical (e.g. infectious disease) illness would be excluded.
20. Patients must not have prolonged QTcF interval >480 msec
21. Patients with known hypersensitivity to cisplatin, gemcitabine or CPI-613, or its inactive components would be excluded.

### Study Drugs, Doses, Routes, Regimen

Phase IB and Phase II Arm A (days 1, 8 Q21 days): Gemcitabine 1000 mg/m² IV (obtained via reconstitution and dilution of gemcitabine hydrochloride lyophilized powder per commercial package insert); Cisplatin 25 mg/m² IV; CPI-613 at dose level or RP2D IV.

Phase II Arm B (days 1, 8 Q21 days): Gemcitabine 1000 mg/m² IV (obtained via reconstitution and dilution of gemcitabine hydrochloride lyophilized powder per commercial package insert); Cisplatin 25 mg/m² IV.

Protocol treatment must start within 14 calendar days of enrollment otherwise the patient will be taken off study. Re-screening is allowed.

Phase IB Dose Levels, Regimen, and Dose Limiting Toxicities (DLTs)

TABLE 1
Phase IB Dose Levels

| Dose | Devimistat | Gemcitabine | Cisplatin |
|---|---|---|---|
| Level | (mg/m²) | (mg/m²) | (mg/m²) |
| 3 | 2000 | 1000 | 25 |
| 2 | 1500 | 1000 | 25 |
| 1* | 1000 | 1000 | 25 |
| −1 | 500 | 800 | 25 |

*starting dose level

TABLE 2
Phase IB Regimen Description

| | | | | Sched- | Cycle |
|---|---|---|---|---|---|
| Agent | Prophylaxis | Dose | Route^{a} | ule^{b} | Length |
| Devimistat | Palonosetron | Per | IV over 115-125 | Days 1 | 3 weeks |
| | 0.25 mg IV + | Dose | minutes before | and 8 | (21 |
| | dexamethasone | Level | gemcitabine | | days) |
| Gemci- | 12 mg PO/IV + | Per | IV over 25-35 | Days 1 | |
| tabine | fosaprepitant | Dose | minutes after | and 8 | |
| | 150 mg IV, OR | Level | devimistat | | |
| Cisplatin | per institutional | Per | IV over 25-65 | Days 1 | |
| | policy | Dose | minutes after | and 8 | |
| | | Level | gemcitabine | | |

^{a}Infusion times may be extended as needed for safety (e.g. infusion reaction occurs). These instances should be documented in the patient medical records.
^{b}Patients may not be treated on study for longer than 2 years

A DLT will be any of the following occurring during the first 3 weeks of therapy, including cycle 2 day 1, attributed (possibly, probably, or definitely) to the drug combination following day 1 treatment and occurring in the 22-day interval as assessed using the NCI CTCAE v5.0.
a. Grade 4 or greater hematological toxicity with the exception of uncomplicated grade 4 leukopenia/neutropenia lasting <7 days)
b. Grade 3 or higher thrombocytopenia with bleeding
c. Grade 3 or greater febrile neutropenia.
d. Grade 3 or greater non-hematological toxicity with the following exceptions:
   i. Grade 3 nausea, vomiting, or diarrhea <72 hours with adequate antiemetic and other supportive care measures,
   ii. Grade 3 fatigue <1 week,
   iii. Grade 3 or higher electrolyte abnormality that lasts <24 to 72 hours, is not clinically complicated, and resolves spontaneously or responds to conventional medical interventions,
   iv. Grade 3 or higher amylase or lipase that is not associated with symptoms or clinical manifestations of pancreatitis.
e. Any death not clearly due to the underlying disease or extraneous causes.
f. Toxicities meeting Hy's law criteria
   With completion of cycle 1, and following DLT determination (yes vs. no) on cycle 2 day 1, cycle 2 may begin.

Phase II Arm A Regimen

TABLE 3
Phase II Arm A Regimen Description

| | | Dose | | | Cycle |
|---|---|---|---|---|---|
| Agent | Prophylaxis | (mg/m²) | Route^{a} | Schedule^{b} | Length |
| Devimistat | Palonosetron | RP2D | IV over 115-125 | Days 1 | 3 weeks |
| | 0.25 mg IV + | | minutes before | and 8 | (21 |
| | dexamethasone | | gemcitabine | | days) |
| Gemcitabine | 12 mg PO/IV + | 1000^{c} | IV over 25-35 | Days 1 | |
| | fosaprepitant | | minutes after | and 8 | |
| | 150 mg IV, OR | | devimistat | | |
| Cisplatin | per institutional | 25^{c} | IV over 25-65 | Days 1 | |
| | policy | | minutes after | and 8 | |
| | | | gemcitabine | | |

^{a}Infusion times may be extended as needed for safety (e.g. infusion reaction occurs). These instances should be documented in the patient medical records.
^{b}Patients may not be treated on study for longer than 2 years
^{c}per commercial package insert

Phase II Arm B Regimen

TABLE 4
Phase II Arm B Regimen Description

| | | Dose | | Sched- | Cycle |
|---|---|---|---|---|---|
| Agent | Prophylaxis | (mg/m²) | Route^{a} | ule^{b} | Length |
| Gemci- | Palonosetron | 1000^{c} | IV over 25-35 | Days 1 | 3 weeks |
| tabine | 0.25 mg IV + | | minutes before | and 8 | (21 |
| | dexamethasone | | cisplatin | | days) |
| Cisplatin | 12 mg PO/IV + | 25^{c} | IV over 25-65 | Days 1 | |
| | fosaprepitant | | minutes after | and 8 | |
| | 150 mg IV, OR | | gemcitabine | | |
| | per institutional | | | | |
| | policy | | | | |

^{a}Infusion times may be extended as needed for safety (e.g. infusion reaction occurs). These instances should be documented in the patient medical records.
^{b}Patients may not be treated on study for longer than 2 years
^{c}per commercial package insert

Toxicities and Dosing Delays/Dose Modifications

Any patient who receives treatment on this protocol will be evaluable for toxicity. Each patient will be assessed for the development of toxicity according to the Time and Events Table (below). Toxicity will be assessed according to the NCI Common Terminology Criteria for Adverse Events (CTCAE), version 5.0. Dose adjustments should be made according to the system showing the greatest degree of toxicity.

TABLE 5
Dose Modifications

| | | | Percentage | Modified | |
|---|---|---|---|---|---|
| Agent | Current Dose | | Decrease | Dose | |
| Gemcitabine | 1000 | mg/m² | 20% | 800 | mg/m² |
| | 800 | mg/m² | 20% | 640 | mg/m² |
| | 640 | mg/m² | 100% | Discontinue | |
| Cisplatin | 25 | mg/m² | 20% | 20 | mg/m² |
| | 20 | mg/m² | 20% | 16 | mg/m² |
| | 16 | mg/m² | 100% | Discontinue | |
| Devimistat | 2000 | mg/m² | 25% | 1500 | mg/m² |
| | 1500 | mg/m² | 33% | 1000 | mg/m² |
| | 1000 | mg/m² | 50% | 500 | mg/m² |

| | | | |
|---|---|---|---|
| 500 | mg/m² | 100% | Discontinue |

All dose reductions will be permanent unless otherwise noted. If more than one toxicity occurs requiring dose reduction, the dose administered should be based on the most severe toxicity. Treatment delay of more than 28 days from last intended therapy will result in treatment discontinuation. If one of the drugs is discontinued due to toxicity attributed to that agent, the patient will be allowed to continue a modified regimen with the remaining study arm agent/s. However, monotherapy with cisplatin is not permitted, though monotherapy with gemcitabine is allowed. Additionally, if both gemcitabine and cisplatin are held on day 1, then also hold devimistat. If both gemcitabine and cisplatin are discontinued, then devimistat should also be discontinued. Investigators should consider dose re-calculation of gemcitabine and/or cisplatin with change in BSA as per standard of care/institutional guidelines. However, a change in BSA by 10% or more requires a dose re-calculation. If a patient experiences neutropenic fever at any point in the treatment cycle, chemotherapy will be delayed until ANC>1,000 and antibiotic treatment of the event is completed. When treatment resumes, proceed with one dose level reduction for one or more agents. Doses will not be modified for cholangitis attributable to biliary obstruction/stent occlusion unless this occurs in the setting of >grade 3 neutropenia. Laboratory abnormalities that are not clinically relevant (i.e., lymphopenia) do not require modification of dosing. Missed Dose: If the regimen held or missed was to be given on Day 1 then that next cycle will not be considered to start until the day the first dose is actually administered. Held doses of either or all drugs on Day 8 will be considered omitted.

TABLE 6
Day 1 Dose Modifications for Hematologic Toxicity

| Hematologic Toxicity | Dose Adjustment |
|---|---|
| ANC^{a} ≥1000/mm³ | Treat as scheduled |
| AND | |
| Platelets ≥100,000/mm | |
| ANC^{a} <1000/mm³ | Hold all protocol treatment up to a maximum |
| OR/AND | of 28 days until ANC ≥1000/mm³ AND |
| Platelets <100,000/mm³ | platelets ≥100,000/mm³ then resume |
| | at next lower dose level for either OR both |
| | gemcitabine and cisplatin as detailed in Table |
| | 5. If not resolved, then discontinue all |
| | treatment. |

^{a}Note:
Growth factors may be added for low ANC BEFORE a dose reduction is instituted at the treating physician's discretion

TABLE 7
Day 8 Dose Modifications for Hematologic Toxicity

| | Dose Adjustment | Dose Adjustment | Dose Adjustment |
|---|---|---|---|
| Hematologic Toxicity | for Gemcitabine | for Cisplatin | for Devimistat |
| ANC ≥1000/mm³ | No change in dose | No change in dose | No change in dose |
| AND | | | |
| Platelets ≥75,000/mm³ | | | |
| ANC 500-999/mm³ | Decrease Day 8 | Decrease Day 8 | No change in dose |
| OR | dose by 1 dose | dose by 1 dose | |
| Platelets | level for this cycle | level for this cycle | |
| 50,000-74,999/mm³ | Consider decrease | Consider decrease | |
| | Day 1 & 8 by 1 | in Day 1 & 8 dose | |
| | dose level for | by 1 dose level for | |
| | subsequent cycles | subsequent cycles | |
| ANC <500/mm³ | Hold Day 8 | Hold Day 8 | Hold Day 8 |
| OR | treatment | treatment | treatment |
| Platelets <50,000/mm³ | Decrease Day 1 | Decrease in Day 1 | Decrease in Day 1 |
| | & 8 by 1 dose | & 8 dose by 1 dose | & 8 dose by 1 dose |
| | level for | level for | level for |
| | subsequent cycles^{a} | subsequent cycles^{a} | subsequent cycles^{a} |

^{a}Note:
Dose level reduction may be for one or more agents as per investigator discretion when toxicity is at least possible attributed to the agent

TABLE 8
Dose Modifications for Non-Hematologic Toxicity

| Non-Hematologic | Dose Adjustment | Dose Adjustment | Dose Adjustment |
|---|---|---|---|
| Toxicity | for Gemcitabine | for Cisplatin | for Devimistat |
| Alopecia, venous | No change in dose | No change in dose | No change in dose |
| thromboembolism | | | |
| Grade ≥3 nausea and | No change in dose | Consider dose | Hold until resolved |
| vomiting (ongoing after | | reduction to next | to Grade 1 or |
| maximal anti-emetic | | dose level or | baseline, and then |
| therapy) | | discontinuation | consider dose |
| | | | reduction to next |
| | | | dose level |
| Grade ≥3 diarrhea (at | No change in dose | Consider dose | Hold until resolved |
| least possibly attributed | | reduction to next | to Grade 1 or |
| after negative infectious | | dose level | baseline, and then |
| work-up AND maximal | | | consider dose |
| supportive therapy) | | | reduction to next |
| | | | dose level |
| Grade ≥2 | Hold all protocol treatment up to a maximum of 28 days until | | |
| Hyperbilirubinemia | toxicity resolves to Grade ≤1 or baseline, then may resume at | | |
| | same doses as before or next dose level for one or more agents. | | |
| | If not resolved, then discontinue all treatment. Doses will not | | |
| | be modified for cholangitis attributable to biliary | | |
| | obstruction/stent occlusion unless this occurs in the setting of | | |
| | grade ≥3 neutropenia | | |
| Grade ≥3 possibly | Hold all protocol treatment up to a maximum of 28 days until | | |
| attributable to cytotoxic | toxicity resolves to Grade ≤1 or baseline, then resume at next | | |
| treatment | dose level for one or more agents. | | |
| ≥Grade 3 Peripheral | No change in dose | Hold up to a | No change in dose |
| neuropathy | | maximum of 28 | |
| | | days until toxicity | |
| | | resolves to Grade ≤2, | |
| | | then consider | |
| | | dose reduction to | |
| | | next dose level. | |
| ≥Grade 2 Creatinine | No change in dose | Hold, assess | Hold, assess |
| increase (>1.5 × | | hydration and | hydration and |
| baseline or ULN | | general medical | general medical |
| whichever is higher) | | status of the | status of the- |
| | | patient. Cisplatin | patient. Devimistat |
| | | may be resumed if | may be resumed if |
| | | creatinine | creatinine |
| | | improves to <1.5 × | improves to <1.5 × |
| | | ULN or baseline | ULN or baseline |
| | | with one dose | with one dose level |
| | | level reduction. | reduction |

Note:
Laboratory abnormalities that are not clinically relevant (i.e., lymphopenia) do not require modification of dosing. All dose adjustments for toxicity will be described in the clinical record.

Concomitant Medications/Treatments. The following concomitant medications or treatments are not permitted while the patient is currently receiving therapy on the protocol:
Other investigational agents
Concurrent radiation

The following concomitant medications may require additional monitoring during cisplatin therapy:
Plasma levels of anticonvulsant agents (valproic acid, phenytoin, and carbamazepine) may become sub-therapeutic and should be monitored.
Concomitant use with aminoglycosides, tacrolimus, and amphotericin B increase risk of nephrotoxicity.
Concomitant use with loop diuretics and aminoglycosides increase risk of ototoxicity.
Concurrent use with lithium may result in reduced lithium plasma concentration.
Concurrent use with warfarin may result in increased INR.
Concurrent use with thioctic acid may result in decreased cisplatin effectiveness and should be avoided.

Duration of Therapy. Treatment may continue for a total of 2 years or until one of the following criteria apply:
Disease progression (as defined below)
Inter-current illness that prevents further administration of treatment
Unacceptable adverse event(s)
Sexually active subjects who refuse to use medically accepted barrier methods of contraception (e.g., male condom, female condom) during the course of the study and for 6 months after discontinuation of study treatment
Patient who becomes pregnant or is breastfeeding
Patient who cannot tolerate the minimum protocol-specified dose of study treatment
Request by regulatory agencies for termination of treatment of an individual subject or all subjects under the protocol
Significant noncompliance with the protocol schedule in the opinion of the investigator
Patient voluntarily withdraws from treatment OR
General or specific changes in the patient's condition render the patient unacceptable for further treatment in the judgment of the investigator

Off Treatment Criteria. Patients will be removed from protocol therapy when any of the criteria listed above under Duration of Therapy apply. Document in the source the reason for ending protocol therapy and the date the patient was removed from treatment. All patients who discontinue treatment should comply with protocol specific follow-up procedures as outlined below under Off Study Criteria. The only exception to this requirement is when a subject withdraws consent for all study procedures or loses the ability to consent freely.

Duration of Follow-Up. After treatment discontinuation, follow-up for survival and initiation of any other anti-cancer therapies will be documented every 3 months via telephone or office visit documentation for up to 2 years from treatment discontinuation or until death, whichever comes first, or 3 years after first date of treatment initiation for those that remain on treatment. Patients removed from treatment for unacceptable adverse events will also be followed more closely until resolution or stabilization of the adverse event.

Off Study Criteria. Patients can be taken off study at any time at their own request, or they may be withdrawn at the discretion of the investigator for safety, behavioral or administrative reasons. The reason(s) for discontinuation from study will be documented and may include:
a. Patient withdraws consent (termination of treatment and follow-up)
b. Loss of ability to freely provide consent through imprisonment or involuntary incarceration for treatment
c. Termination of the study by the Sponsor, the site, or the FDA
d. Patient completes protocol treatment and follow-up criteria.

Patient Replacement. All patients who receive at least one dose of study therapy will be considered evaluable. Patients enrolled in the study will be considered non-evaluable under the following case scenarios and replaced by additional patients:
a. Patients who received no investigational therapy
b. Patients who were discontinued or withdrew consent for study therapy prior to first response evaluation.
c. Patients meeting Off Study Criteria a. and b. (above).

### Measurement of Effect

Antitumor Effect-Solid Tumors. Objective response assessment will be determined by review of CT or MR scans of the chest, abdomen and pelvis using RECIST v1.1 every 8 weeks+/−1 week while patients are on treatment (Eisenhauer, E. A., P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe and J. Verweij, “New response evaluation criteria in solid tumours: revised RECIST guideline (version 1.1),” Eur J Cancer, 2009, 45(2): 228-247).

### Definitions

Evaluable for toxicity. All patients will be evaluable for toxicity from the time of their first treatment with study drug.
Evaluable for phase IB primary endpoint, MTD/RP2D. All patients that receive at least one dose of study therapy will be considered evaluable. Patients enrolled to therapy but that never receive study therapy will be replaced. (See Patient Replacement, above).
Evaluable for phase II primary endpoint, overall response rate. All enrolled patients who received at least 1 cycle(s) of therapy, and had their disease re-evaluated will be considered evaluable for response. These patients will have their response classified according to the definitions stated below. (Note: Patients who exhibit objective disease progression prior to the end of cycle 1 will also be considered evaluable.)

Disease Parameters

Measurable disease. Measurable lesions are defined as those that can be accurately measured in at least one dimension (longest diameter in the plane of measurement is to be recorded) with a minimum size of:
10 mm by CT scan (irrespective of scanner type) for studies with a slice thickness of ≤5 mm or twice the slice thickness or MRI
10 mm caliper measurement by clinical exam (lesions which cannot be accurately measured with calipers should be recorded as non-measurable)
20 mm by chest X-ray (if clearly defined and surrounded by aerated lung)

All tumor measurements must be recorded in millimeters (or decimal fractions of centimeters).

Malignant lymph nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

Note: Tumor lesions that are situated in a previously irradiated area will only be considered measurable, if they have had subsequent progression by at least 5 mm.

Non-measurable disease. All other lesions (or sites of disease), including small lesions (longest diameter <10 mm using CT scan), are considered non-measurable disease. Bone lesions without measurable soft tissue component, leptomeningeal disease, ascites, pleural/pericardial effusions, lymphangitis cutis/pulmonitis, inflammatory breast disease, abdominal masses (not followed by CT or MRI), and cystic lesions are all non-measurable.

Target lesions. All measurable lesions up to a maximum of 2 lesions per organ and 5 lesions in total should be identified as target lesions and recorded and measured at baseline. Target lesions should be selected on the basis of their size (non-nodal lesions with the longest diameter), be representative of all involved organ(s), but in addition should be those that lend themselves to reproducible repeated measurements.

Lymph nodes merit special mention since they are normal anatomical structures that may be visible by imaging even if not involved by tumor. Pathological nodes that are defined as measurable and may be identified as target lesions must meet the criterion of a short axis of >15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, sagittal or coronal). The smaller of these measures is the short axis. For example, an abdominal node which is reported as being 20 mm×30 mm has a short axis of 20 mm and qualifies as a malignant, measurable node. In this example, 20 mm should be recorded as the nodal measurement. All other pathological nodes (those with short axis ≥10 mm but <15 mm) should be considered non-target lesions. Nodes that have a short axis <10 mm are considered non-pathological and should not be recorded or followed. A sum of the diameters (longest for non-nodal lesions, short axis for nodal lesions) for all target lesions will be calculated and reported as the baseline sum of diameters. If lymph nodes are to be included in the sum, then as noted above, only the short axis is added into the sum. The baseline sum of diameters will be used as reference to further characterize any objective tumor regression in the measurable dimension of the disease.

Non-target lesions. All other lesions (or sites of disease) including pathological lymph nodes should be identified as non-target lesions and should also be recorded at baseline. Measurements are not required, and these lesions should be followed as ‘present’, ‘absent’, or in rare cases ‘unequivocal progression.’ In addition, it is possible to record multiple non-target lesions involving the same organ as a single item on the case record form (e.g. ‘multiple enlarged pelvic lymph nodes’ or ‘multiple liver metastases’).

Guidelines for Evaluation of Measurable Disease
All measurements should be recorded in metric notation, using calipers if clinically assessed. All baseline evaluations should be performed as close as possible to the treatment start date and never more than 4 weeks before the beginning of the treatment.
The same method of assessment and the same technique should be used to characterize each identified and reported lesion at baseline and during subsequent follow-up studies Imaging-based evaluation should always be done rather than clinical examination unless the lesion(s) being followed cannot be imaged but are assessable by clinical exam.
Clinical lesions: Clinical lesions will only be considered measurable when they are superficial and >10 mm diameter as assessed using calipers (e.g. skin nodules). For the case of skin lesions, documentation by color photography including a ruler to estimate the size of the lesion is suggested. As noted above, when lesions can be evaluated by both clinical exam and imaging, imaging evaluation should be undertaken since it is more objective and may also be reviewed at the end of the study.
CT, MRI: CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (e.g. for body scans).
Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next. If new lesions are identified by ultrasound in the course of the study, confirmation by CT or MRI is advised. If there is concern about radiation exposure at CT, MRI may be used instead of CT in selected instances.
Endoscopy, laparoscopy: The utilization of these techniques for objective tumor evaluation is not advised. However, they can be useful to confirm complete pathological response when biopsies are obtained or to determine relapse in trials where recurrence following complete response or surgical resection is an endpoint.
Tumor markers: Tumor markers alone cannot be used to assess objective tumor response.

Response Criteria

Evaluation of Target Lesions
Complete Response (CR): Disappearance of all target lesions, determined by two separate observations conducted not less than 4 weeks apart. There can be no appearance of new lesions.
Partial Response (PR): At least a 30% decrease in the sum of the longest diameter (LD) of target lesions, taking as reference the baseline sum LD. There can be no appearance of new lesions.
Progressive Disease (PD): At least a 20% increase in the sum of the LD of target lesions (with a minimum absolute increase of 5 mm), taking as reference the smallest sum LD recorded since the treatment started, or the appearance of one or more new lesions.
Stable Disease (SD): Neither sufficient shrinkage to qualify for PR (taking as reference the baseline sum LD) nor sufficient increase to qualify for PD (taking as reference the smallest sum LD since the treatment started).

Evaluation of Non-Target Lesions
Complete Response (CR): Disappearance of all non-target lesions. All lymph nodes should be non-pathological in size (<10 mm short axis)
Non-CR/Non-PD: Persistence of one or more non-target lesion(s)
Progressive Disease (PD): Appearance of one or more new lesions and/or unequivocal progression of existing non-target lesions.
Although a clear progression on non-target lesions in absence of stable target lesions is exceptional, the opinion of the treating physician should prevail in such circumstances

Evaluation of the Best Overall Response. The best overall response is the best response recorded from the start of the treatment until disease progression/recurrence (taking as reference for progressive disease the smallest measurements recorded since the treatment started). The patient's best response assignment will depend on the achievement of both measurement and confirmation criteria. Note: If subjects respond to treatment and are able to have their disease resected, the patient's response will be assessed prior to the surgery.

TABLE 9
Response Evaluation as per RECISTv1.1

| | | | Overall | |
|---|---|---|---|---|
| | Non- | | Response | Confirmed Response |
| Target | Target | New | per | for this |
| Lesions | Lesions | Lesions | RECISTv1.1 | Category Requires: |
| CR | CR | No | CR | ≥4 weeks confirmation |
| CR | CR | No | PR | ≥4 weeks confirmation |
| | Non-CR/PD | | | |
| PR | CR | No | | |
| | Non-CR/PD | | | |
| SD | CR | No | SD | Documented at least |
| | Non-CR/PD | | | once ≥4 weeks from |
| | | | | baseline |
| PD | Any | Any | PD | ≥4 weeks confirmation |
| Any | PD* | Any | | |
| Any | Any | No | | |

*Only in exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.

Duration of Response
Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).
Duration of stable disease: Stable disease is measured from the start of the treatment until the criteria for progression are met, taking as reference the smallest measurements recorded since the treatment started.

Safety/Tolerability. Analyses will be performed for all patients having received at least one dose of study drug. The study will use the CTCAE version 5.0 for reporting of adverse events (http://ctep.cancer.govireporting/ctc.html).

### Adverse and Other Reportable Events

Adverse Event Reporting Requirements.

Adverse event (AE) monitoring and reporting is a routine part of every clinical trial and is done to ensure the safety of subjects enrolled in the studies as well as those who will enroll in future studies using similar agents. Data on adverse events will be collected from the time of the initial study treatment administration through 100 days after the last dose of study treatment. Any serious adverse event that occurs more than 100 days after the last study treatment and is considered related to the study treatment or intervention must also be reported. Serious Adverse Events (SAEs) will continue to be followed until:
Resolution or the symptoms or signs that constitute the serious adverse event return to baseline;
There is satisfactory explanation other than the study treatment for the changes observed; or
Death.

The investigator is responsible for the detection, documentation, grading and assignment of attribution of events meeting the criteria and definition of an AE or SAE. The definitions of AEs and SAEs are given below. It is the responsibility of the principal investigator to ensure that all staff involved in the trial is familiar with the content of this section.

Any medical condition or laboratory abnormality with an onset date before initial study treatment administration is considered to be pre-existing in nature. Any known pre-existing conditions that are ongoing at time of study entry should be considered medical history.

All events meeting the criteria and definition of an AE or SAE (as defined below under “Adverse Event Characteristics”) occurring from the initial study treatment administration through 100 days following the last dose of the study treatment must be recorded as an adverse event in the patient's source documents and on the CRF regardless of frequency, severity (grade) or assessed relationship to the study treatment or intervention. However, with regards to laboratory and vital sign abnormalities, only those which require protocol treatment to be modified or treatment to be rendered should be reported as AEs

In addition to new events, any increase in the frequency or severity (i.e., toxicity grade) of a pre-existing condition that occurs after the patient begins study treatment is also considered an adverse event. However, anticipated fluctuations of pre-existing conditions, including the disease under study, that don't represent a clinically significant exacerbation or worsening, need not be reported as AEs.

### Definitions

Adverse Event. An adverse event (AE) is any untoward medical occurrence in a patient receiving study treatment and which does not necessarily have a causal relationship with this treatment. An AE can be any unfavorable and unintended sign (including an abnormal laboratory finding), symptom, or disease temporally associated with the use of an experimental intervention, whether or not related to the intervention.

Diagnostic and therapeutic non-invasive and invasive (i.e., surgical) procedures will not be reported as adverse events. However, the medical condition for which the procedure was performed must be reported if it meets the definition of an adverse event unless it is a pre-existing (prior to protocol treatment) condition.

Serious Adverse Event. An adverse event is considered “serious” if, in the view of either the investigator or sponsor-investigator, it results in any of the following outcomes:
Death
If death results from (progression of) the disease, the disease should be reported as event (SAE) itself.
A life-threatening adverse event
An adverse event is considered ‘life-threatening’ if, in the view of either the investigator [or sponsor], its occurrence places the patient or subject at immediate risk of death. It does not include an adverse event that, had it occurred in a more severe form, might have caused death.
Inpatient hospitalization or prolongation of existing hospitalization for >24 hours
A persistent or significant incapacity or substantial disruption of the ability to conduct normal life functions
A congenital anomaly/birth defect
Important medical event
Any event that may not result in death, be life-threatening, or require hospitalization may be considered serious when, based upon appropriate medical judgment, they may jeopardize the patient and may require medical or surgical intervention to prevent one of the outcomes listed in this definition of “Serious Adverse Event.” Examples of such medical events include allergic bronchospasm requiring intensive treatment in an emergency room or at home; convulsions that do not result in inpatient hospitalization or the development of drug dependency or drug abuse.

Previously planned (prior to signing the informed consent form) surgeries should not be reported as SAEs unless the underlying medical condition has worsened during the course of the study. Preplanned hospitalizations or procedures for preexisting conditions that are already recorded in the patient's medical history at the time of study enrollment should not be considered SAEs. Hospitalization or prolongation of hospitalization without a precipitating clinical AE (for example, for the administration of study therapy or other protocol-required procedure) should not be considered SAEs. However, if the preexisting condition worsened during the course of the study, it should be reported as an SAE.

Expected Adverse Events. An adverse event (AE) is considered “expected” if:
For approved and marketed drugs or devices, those adverse events are described in the approved Package Insert (Label).
For investigational new drugs or devices, those adverse events are described in the devimistat Investigator's Brochure.
In clinical research studies, information on expected adverse events is also summarized in the protocol and in the consent document.

Unexpected Adverse Event. An adverse event (AE) is considered “unexpected” if it is not described in the Package Insert, devimistat Investigator's Brochure, in published medical literature, in the protocol, or in the informed consent document.

Adverse Event Characteristics

CTCAE Term. (AE description) and grade: The descriptions and grading scales found in the NCI Common Terminology Criteria for Adverse Events (CTCAE) version 5.0 will be utilized for AE reporting. All appropriate treatment areas should have access to a copy of the CTCAE version 5.0. A copy of the CTCAE version 5.0 can be down loaded from the CTEP web site. (http://ctep.cancer.gov)

Attribution of the AE. The investigator or co-investigator is responsible for assignment of attribution.
RELATED
   Definite—The AE is clearly related to the study treatment.
   Probable—The AE is likely related to the study treatment.
   Possible—The AE may be related to the study treatment.
UNRELATED
   Unlikely—The AE is doubtfully related to the study treatment.
   Unrelated—The AE is clearly NOT related to the study treatment/intervention.

Serious Adverse Event Reporting Guidelines

Reporting Procedures for Multi-Site Trials. All serious adverse events (SAEs) and unanticipated problems (UPs), regardless of causality to study drug, will be reported to the Principal Investigator and also to the Coordinating Center. All SAEs and UPs must be reported to the Coordinating Center within 24 hours of first awareness of the event. Events should be reported using the Coordinating Center's SAE form as available in the study database. A copy of the SAE form as available in the study database should be sent to the Coordinating Center via fax or email within 24 hours of the site's knowledge of the event.

Follow-up information must also be reported within 24 hours of receipt of the information by the investigator.

All SAEs and UPs will be reported to the IRB per current institutional standards

The Coordinating Center will disseminate information regarding SAEs and UPs to the participating sites within 5 days of review of the information by the Coordinating Center's Principal Investigator (or designee in the event of extended absence) only in the case that the event(s) is believed to be related (i.e., possibly, probably, or definitely) to the study drug. The Coordinating Center will be responsible for reporting of events to the FDA and supporters, as appropriate (outlined below).

Reporting Procedures to Sponsor. All Serious Adverse Events (SAEs) occurring from the initial study treatment administration through 100 days following the last dose of the study treatment will be reported by the Coordinating Center to the study sponsor. Any SAEs occurring after 100 days following the last dose of the study treatment that are believed to be related to study drug will also be reported to the sponsor.

The Coordinating Center will send the initial completed SAE Form within 24 hours of receipt via email to the sponsor.

If only limited information is initially available or an ongoing SAE changes in its intensity or relationship to the study drug, or if new information becomes available, a follow-up report will be generated and sent to the sponsor within 24 hours of receipt.

Reporting Procedures to FDA. In this trial, serious, unexpected adverse events believed to be definitely, probably or possibly related to the study treatment will be reported to the Food and Drug Administration via the MedWatch 3500A Form. The site's Assistance Program will assist the IND Sponsor in reporting SAEs to the FDA that meet the reporting requirements in 21 CFR 312.32. This reporting could include the initial report and follow-up reports when appropriate for the event. SAEs not meeting the expedited reporting requirements will be submitted to the FDA with the IND Annual Report.

Routine Reporting. All other adverse events—such as those that are expected, or are unlikely or definitely not related to the study participation—are to be reported annually as part of regular data submission.

Reporting of Pregnancy. Pregnancies that occur during study participation or within 6 months of last study dose should be reported to the Coordinating Center via e-mail immediately upon site's knowledge of the event.

Reporting of Unanticipated Problems. There are types of incidents, experiences and outcomes that occur during the conduct of human subjects research that represent unanticipated problems but are not considered adverse events. For example, some unanticipated problems involve social or economic harm instead of the physical or psychological harm associated with adverse events. In other cases, unanticipated problems place subjects or others at increased risk of harm, but no harm occurs.

Upon becoming aware of any incident, experience, or outcome (not related to an adverse event) that may represent an unanticipated problem, the investigator should assess whether the incident, experience, or outcome represents an unanticipated problem. The incident, experience or outcomes is considered unanticipated if it meets all of the following criteria:
Unexpected (in terms of nature, severity, or frequency);
Related or possibly related to participation in the research; and
Suggests that the research places subjects or others at a greater risk of harm than was previously known or recognized.

If the investigator determines that the incident, experience, or outcome represents an unanticipated problem, the investigator must report it to the IRB within 14 calendar days of the study team becoming aware of the problem.

Safety Report Reconciliation. The sponsor will reconcile the clinical database SAE reports transmitted to the sponsor. Frequency of reconciliation should be every 3 months and prior to the database lock or final data summary. The sponsor will email, upon request from the Investigator, the GPV&E reconciliation report. The data elements listed on the GPV&E reconciliation report will be used for identification purposes. If the Investigator determines a report was not transmitted to the sponsor, the report should be sent immediately.

### Statistical Considerations

Study Design/Study Endpoints. This protocol will enroll patients with advanced, unresectable BTC to receive gemcitabine, cisplatin and devimistat in a limited Phase 1b study to confirm the safety profile of the combination therapy and the recommended Phase 2 dose (RP2D). The primary endpoint for the phase 1b portion is the occurrence/lack thereof of dose-limiting toxicity (DLT, defined above) during the first 21-day cycle of combination therapy. After determining the combination RP2D additional patients will be enrolled in the phase 2 portion of the study and randomized 2:1 to treatment arms A (combination regimen at RP2D) and B (gemcitabine and cisplatin alone), respectively. We will stratify the randomization by locally advanced versus metastatic disease. The primary endpoint for the phase 2 study is the ORR (PR+CR) per RECIST v1.1 criteria during active study treatment. Secondary endpoints include PFS and OS. The statistical comparison will not be between treatment groups; rather between the experimental treatment arm (A) and historical ORR levels. We will use the estimated ORR for the limited-sized control arm (B) to confirm the control arm has similar response rate as the historical ORR value.

Phase 1B. We will implement a limited phase 1b trial to assure that the dose levels suggested for this combination are safe. Because toxicity across the combined agents is expected to be mildly additive and not synergistic, and in light of the poor prognosis for these patients, we are willing to tolerate up to but not exceeding 35% DLT toxicity proportion during the first cycle (21-days) of therapy. To investigate this, we will enroll up to 20 patients with dose allocation using the Time-to-Event (TiTE) (Cheung, Y. K. and R. Chappell, “Sequential designs for phase I clinical trials with late-onset toxicities,” Biometrics, 2000, 56(4), 1177-1182; O'Quigley, J., M. Pepe and L. Fisher, “Continual reassessment method: a practical design for phase 1 clinical trials in cancer,” Biometrics, 1990, 46(1), 33-48). This method assumes a model for the time to occurrence of toxic responses as a function of the dose. The time-to-event modification allows information from all patients treated (even those with only partial observation) to contribute information for calculating the dose-toxicity relationship. This method is flexible with regard to the number of patients treated at each dose level, patients may be continuously recruited without recruitment pauses subject to the trial's explicit accrual rules, given patients are assigned to a dose level deemed safe at the time of her/his enrollment. The dose toxicity relationship is estimated using all toxicity data observed when each new patient is scheduled to begin treatment. Population for estimation: Every patient enrolled and receiving at least one dose of devimistat will be considered evaluable for estimation of the probability of DLT. If a patient withdraws for any reason during the first cycle of therapy, not primarily or secondarily associated with toxicity, that patient's follow-up will be used in the ongoing estimation of the probability of toxicity; however, such a patient would be replaced when calculating the maximum trial size. Patients stopping treatment early due to or secondary to toxicity will be considered to have had a DLT event (see DLT definition above).

DLT target rate to determine MTD/RP2D. The highest probability of DLT at a dose level acceptable during the first cycle of therapy is 35%. The dose level with estimated probability closest to, but not exceeding the DLT target rate will be considered the RP2D.

Criterion for dose escalation. The dose level will be assigned to each new patient according to the estimates from the TiTE-CRM algorithm. Whenever a patient presents for enrollment on this trial, the probability of DLT will be estimated for each dose level, based upon the initial expectations and the incidence of DLT events in patients already treated on trial weighted by the percentage of the acute DLT observation period observed. The level that has an estimated toxicity closest to the target rate for DLT will be assigned, subject to the following:
The dose level cannot be escalated until at least 2 patients have been observed for the entire DLT observation period (22 days).
Patient 1 and 2 will be assigned to receive dose level 1.
Dose escalation is restricted to one dose level between consecutively treated patients.
Dose de-escalation is not restricted and may be reduced by more than one level between consecutively treated patients.
If at enrollment the TiTE-CRM algorithm estimates all dose levels (−1, 1, 2, and 3) to have probability of DLT above our target level (35%), the trial will close, declaring all available dose levels too toxic for treatment. The trial may seek to remain open after initiating a protocol amendment to alter dose levels to be less toxic.

Patients will be recruited and treated on this trial as naturally available. Accrual is estimated to be approximately 2 patients per month. The operating characteristics of this Phase 1B trial were evaluated through simulation using estimate for the true probability of DLT at each dose level under 3 scenarios (n=1,000 simulated trials per scenario). In the first scenario, the true probability of DLT at each dose level is as we had expected a posteriori; the second scenario we have moderately underestimated the true probabilities of DLT for higher dose levels; the third scenario the additive effect of devimistat upon the observed toxicity from the chemotherapy regimen is negligible and not dose dependent. The dose level nearest to but not exceeding our target rate is bolded for each scenario.

TABLE 10
Operating Characteristics of the Phase 1B Trial

| Dose | Expected | Simulation | Simulation | Simulation |
|---|---|---|---|---|
| Level | Probablity of DLT | Scenario #1 | Scenario #2 | Scenario #3 |
| −1 | 0.20 | 0.20 | 0.20 | 0.20 |
| 1 | 0.25 | 0.25 | 0.30 | 0.21 |
| 2 | 0.30 | 0.30 | 0.40 | 0.22 |
| 3 | 0.35 | 0.35 | 0.50 | 0.23 |

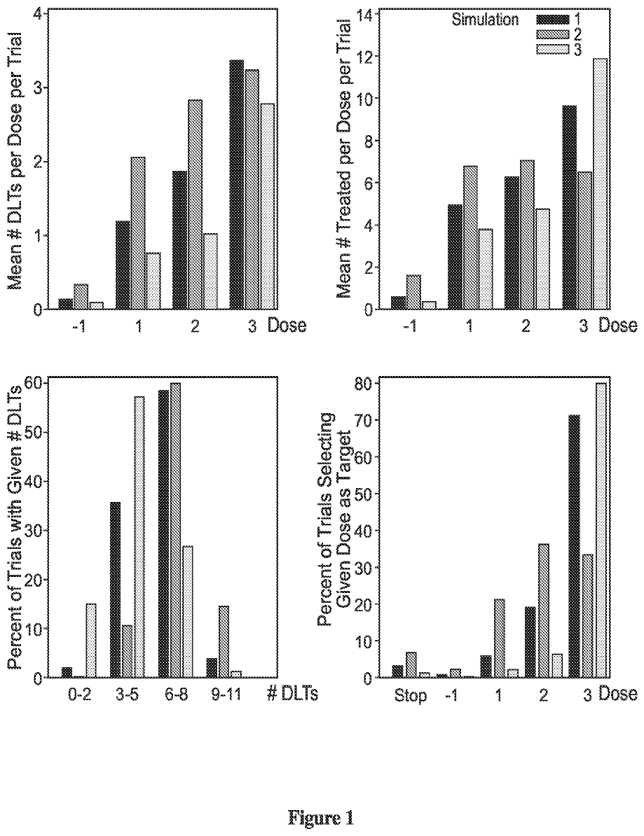

FIG. 1 illustrates (top left) the mean number of patients per trial experiencing a DLT per dose level per simulated scenario; (top right) the mean number of patient per trial treated per dose level per simulated scenario; (bottom left) percent of simulated trials per simulated scenario that had 0-2, 3-5, 6-8, and 9-11 patients in total experiencing DLT events; (bottom right) the percent of simulated trials per simulated scenario that picked dose level −1, 1, 2, or 3 as the MTD or that declared all level too toxic and stopped the trial early.

Reviewing the operating characteristics suggest that when the true probability of DLT is as expected (simulation scenario #1), the trial design will treat nearly 10 patients at the target level dose and selects that dose correctly as the MTD/RP2D dose in over 70% of simulated trials. Similarly, if toxicity is not increased by adding devimistat to SoC chemotherapy (simulation scenario #3), the design treats nearly 12 patients at the target level dose and selects that dose as the MTD/RP2D in approximately 80% of simulated trials. In the event that we have underestimated toxicity a posteriori and the probability of DLT increases more sharply with increasing dose of devimistat, the design will pick a dose at or below the target level in the majority of simulated trials more likely to pick lower dose levels or stop early.

Phase 2. Upon completion of the 20 patient Phase 1b portion of the trial, an MTD/RP2D will have been determined or all dose levels for the combination deemed excessively toxic. After the MTD/RP2D is determined, additional patients will be enrolled and randomized in a 2 (Arm A: devimistat+gemcitabine+cisplatin) to 1 (Arm B: gemcitabine+cisplatin) manner during the phase 2 portion of this trial to assess efficacy as measured by the ORR. Historical data suggest the ORR for Arm B in this patient population is 25% (Valle, Wasan et al. 2010). We suggest that the combination therapy at the MTD/RP2D would be successful by increasing the ORR to 43%. In order to have at least 80% power to detect such a difference significantly, with at most 5% type one error (using a one-sided test of proportions), 50 patients receiving the combination therapy at the MTD/RP2D are necessary.

The operating characteristics presented above for the Phase 1B portion of the trial suggest that 10-12 patients will have been treated at the MTD/RP2D and those patients will be included in the Phase 2 efficacy assessment. Therefore, the Phase 2 portion of the trial will randomize approximately 48 to 58 patients in order to assure 50 patients in the devimistat treated group. Approximately 16 to 20 patients will be assigned to Arm B/standard of care regimen.

The comparison of interest will be between the observed ORR in those receiving combination therapy versus the historical estimate. Patients randomized to Arm B will help suggest whether the historical estimate for ORR is a reasonable null hypothesis for comparison. If the ORR estimated for the control group is higher than the historical value, it may suggest increasing the null hypothesis value for comparison. In order to operationalize this comparison, we will implement a Bayesian approach to estimate the probability of ORR (CR+PR) for the control patients. Given that the historical estimates published by Valle, et. al 2010 is −25% for the 160 patients treated in that phase 3 trial, we will formalize that probability using a beta distribution b (40,120), which is a unimodal distribution, with mean 0.25 with precision proportional to the sample size, 160. We will then collect binary outcome data for the control patients (yes/no ORR) which allows the posterior distribution (prior data+trial's experience for control patients) to be described also as a beta distribution. If after adding the trial's control data it is suggested that the probability that the ORR is greater than 25% is more than 0.7 (70%), we will increase the estimate for the null hypothesis in the primary outcome comparison to be the probability estimated from the mean of the posterior distribution. The following table lists the number of control patients with an overall response based upon the number of control patients treated (may range from 16 to 20) for biologically reasonable ORR rates above 25%.

TABLE 11
Operating Characteristics of the Control Arm in Phase 2

| | | Null Hypothesis value for the |
|---|---|---|
| # of Control | # of Control | Probability of ORR for control |
| Cases Randomized | Cases with | patients used to compare to |
| and Treated | an OR | experimental treatment |
| 16 | 8 | 0.27273 |
| 16 | 9 | 0.27841 |
| 17 | 8 | 0.27119 |
| 17 | 9 | 0.27684 |
| 17 | 10 | 0.28249 |
| 18 | 8 | 0.26966 |
| 18 | 9 | 0.27528 |
| 18 | 10 | 0.28090 |
| 19 | 8 | 0.26816 |
| 19 | 9 | 0.27374 |
| 19 | 10 | 0.27933 |
| 19 | 11 | 0.28492 |
| 20 | 9 | 0.27222 |
| 20 | 10 | 0.27778 |
| 20 | 11 | 0.28333 |

Data Analyses Plans. We will report the estimate probability of DLT with 95% Bayesian Credible Intervals for each dose level as estimated by the TiTE-CRM algorithm at the completion of the Phase 1. The probability of DLT will be updated using the experience of the Phase 2 patients at the MTD/RP2D as well.

We will record ORR as a yes/no outcome for each patient for the period of active study treatment and report the estimate and exact 95% binomial confidence intervals. We will estimate PFS and OS using the product-limit method of Kaplan and Meier. Follow-up time will be defined as time from date of first study treatment until the date of radiological or clinical progression (leading to withdrawal from the study), or death from any cause, whichever comes first for PFS and for only death from any cause for OS. For patients without events, we will censor the follow-up time at the date of last disease evaluation at the time of analysis. We will report estimates for the median and 75th percentiles with 95% confidence intervals. We will summarize additional safety data (e.g., laboratory safety parameters, vital signs, concomitant medications and new physical examination findings) descriptively by reporting counts and percentages, with exact binomial confidence intervals where appropriate. ORR will be determined as per the RECISTv1.1 guidelines. We will report adverse events per the NCI CTCAE v5.0.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating biliary tract cancer in a patient in need thereof, comprising the step of intravenously administering to the patient a therapeutically effective amount of (i) devimistat, (ii) gemcitabine, and (iii) cisplatin, in order to treat the biliary tract cancer.

2. The method of claim 1, wherein the biliary tract cancer is advanced unresectable biliary tract cancer.

3. The method of claim 1, wherein the biliary tract cancer is metastatic.

4. The method of claim 1, wherein the devimistat, gemcitabine, and cisplatin are administered to the patient on days 1 and 8 of a 21 day cycle.

5. The method of claim 4, wherein the devimistat is administered to the patient at a daily dose of about 500 mg/m² to about 2000 mg/m² on days 1 and 8 of the 21 day cycle.

6. The method of claim 5, wherein the devimistat is administered to the patient at a daily dose of about 500 mg/m².

7. The method of claim 5, wherein the devimistat is administered to the patient at a daily dose of about 1000 mg/m².

8. The method of claim 5, wherein the devimistat is administered to the patient at a daily dose of about 1500 mg/m².

9. The method of claim 5, wherein the devimistat is administered to the patient at a daily dose of about 2000 mg/m².

10. The method of claim 4, wherein the gemcitabine is administered to the patient at a daily dose of about 640 mg/m² to about 1000 mg/m² on days 1 and 8 of the 21 day cycle.

11. (canceled)

12. (canceled)

13. (canceled)

14. The method of claim 4, wherein the cisplatin is administered to the patient at a daily dose of about 16 mg/m² to about 25 mg/m² on days 1 and 8 of the 21 day cycle.

15. (canceled)

16. (canceled)

17. (canceled)

18. A method for treating biliary tract cancer in a patient in need thereof, comprising the step of intravenously administering to the patient on days 1 and 8 of a 21 day cycle, a daily dose of (i) 500 mg/m² to 2000 mg/m² devimistat, (ii) 1000 mg/m² gemcitabine, and (iii) 25 mg/m² cisplatin, in order to treat the biliary tract cancer.

19. The method of claim 18, wherein the daily dose of devimistat is 500 mg/m².

20. The method of claim 18, wherein the daily dose of devimistat is 1000 mg/m².

21. The method of claim 18, wherein the daily dose of devimistat is 1500 mg/m².

22. The method of claim 18, wherein the daily dose of devimistat is 2000 mg/m².

23. The method of claim 18, wherein the gemcitabine is administered intravenously to the patient after devimistat has been administered intravenously to the patient.

24. The method of claim 23, wherein the cisplatin is administered intravenously to the patient after gemcitabine has been administered intravenously to the patient.

25. The method of claim 24, wherein the devimistat is administered intravenously to the patient in the form of a pharmaceutical composition comprising devimistat, water, and triethanolamine.

26. The method of claim 25, wherein the pharmaceutical composition comprises devimistat at a concentration of 50 mg/mL and triethanolamine at a concentration of 1M.

27. The method of claim 26, wherein the pharmaceutical composition further comprises dextrose.

28. The method of claim 24, wherein the gemcitabine is administered intravenously to the patient in the form of a pharmaceutical composition comprising gemcitabine hydrochloride and water.

29. The method of claim 5, wherein the gemcitabine is administered to the patient at a daily dose of about 640 mg/m² to about 1000 mg/m² on days 1 and 8 of the 21 day cycle.

30. The method of claim 5, wherein the cisplatin is administered to the patient at a daily dose of about 16 mg/m² to about 25 mg/m² on days 1 and 8 of the 21 day cycle.

31. The method of claim 29, wherein the cisplatin is administered to the patient at a daily dose of about 16 mg/m² to about 25 mg/m² on days 1 and 8 of the 21 day cycle.
